(19) United States
(12) Patent Application Publication
(10) Pub. No.: US 2016/0008280 A1
(43) Pub. Date: Jan. 14, 2016

(54) Dripless Olive Oil for Topical Use

(71) Applicant: Penelope Good, Port Washington, NY (US)

(72) Inventor: Penelope Good, Port Washington, NY (US)

(21) Appl. No.: 14/794,633

(22) Filed: Jul. 8, 2015

Related U.S. Application Data
(60) Provisional application No. 62/024,009, filed on Jul. 14, 2014.

(51) Int. Cl. A61K 9/06; A61K 9/00; A61K 36/63

(52) U.S. Cl. CPC A61K 9/06; A61K 36/63; A61K 9/0014

(57) ABSTRACT

Dripless Olive Oil for Topical Use is a product with similar consistency to an ointment or “Vaseline Petroleum Jelly”. The formula consists of 89-90% oil for scent. The mixture is simultaneously heated and agitated, the agitation continues through cooling producing a semi solid form of the olive oil product. The unique nature of the product is in the use of pure olive oil as the main ingredient. The usefulness of the product is in its ability to aid in the removal of head lice and nits from hair and its use as a natural alternative to petroleum based skin care and maintenance products.

Dripless Olive Oil for Topical Use is a semi solid form of pure olive oil. The pure olive oil is mixed with one or two types of beeswax resulting in a semi-solid consistency. Olive oil in this semi solid consistency can be used as an aid to lice removal, as a moisture barrier and/or an alternative to any petroleum based skin care product. While liquid olive oil may also be utilized for lice and nit removal and as a moisture barrier and skin care product, the liquid consistency is less desirable. Dripless Olive Oil for Topical Use provides the consumer with the benefits of natural olive oil without the undesirable mess to clothing and environment.

Many products currently on the market display olive oil as a listed ingredient, Dripless Olive Oil is truly a natural olive oil product. The pure olive oil content of this product is 89-90%. The product contains 3-5 ingredients. Pure olive oil, polygycerol-3 beeswax, beeswax and essential oil of peppermint or essential oil of lavender, acetone.

The formula establishes Dripless Olive Oil for Topical Use as a basic moisture barrier and healthy alternative to any petroleum based skin products as well as an alternative to liquid olive oil currently being used to help remove lice and nits. The essential oil in the formula is for scent only and not essential accounting for only 0.50%.

The consistency of the Dripless Olive Oil for Topical Use is achieved by heating oil to 80 Degrees Celcius, slowly add Polyglycerol Beeswax and Beeswax if required. Mix for 5 minutes at 80 Degrees Celcius and then turn off heat. Continue agitating on high through cooling phase without entrapment of bubbles. The semi solid consistency makes the Dripless Olive Oil for Topical Use unique. All of the benefits associated with using a natural olive oil remain intact without the dripping and messiness. This formula is unique in its effective natural thickening of pure olive oil.

1. Dripless Olive Oil for Topical Use provides a natural alternative to liquid olive oil with a consistency useful for removal of head lice and nits from hair. The Dripless Olive Oil allows the lice treatment individual to safely apply the Dripless Olive Oil to the lice infested person without the danger of the oil getting into the eyes, ears or dripping onto clothing and household furnishings. The smooth consistency makes the use of any hair combing implement simple and detangling of the hair easy. This allows for easier removal of head lice and their nits both by hand and with the use of combs. The spearmint or lavender scent covers the olive smell sometimes considered disagreeable to children.

2. Dripless Olive Oil for Topical Use is natural alternative to petroleum products used as moisture barriers and skin maintenance products. The Dripless Olive Oil can be used topically as a diaper area moisture barrier. The dripless nature of the mixture and the 89-90% olive oil formula provides a natural alternative to emollient products used on babies and children of all ages and adults as well. The dripless nature of the olive oil makes it useful as a skin softening product as well. The unique aspect is in the 89-90% pure olive oil component of the Dripless Olive Oil for Topical Use formula. It is mostly pure olive oil.
